(19) United States
(12) Patent Application Publication
(10) Pub. No.: US 2024/0207654 A1
(43) Pub. Date: Jun. 27, 2024

(54) DESIGN AND FABRICATION OF THERAPEUTIC ULTRASOUND TRANSDUCER WITH ARBITRARILY SHAPED, DENSELY PACKING, REMOVABLE MODULAR ELEMENTS

(71) Applicant: THE REGENTS OF THE UNIVERSITY OF MICHIGAN, Ann Arbor, MI (US)

(72) Inventors: Zhen XU, Ann Arbor, MI (US); Timothy Lewis HALL, Ann Arbor, MI (US); Jonathan E. LUNDT, Ann Arbor, MI (US)

(21) Appl. No.: 18/555,683

(22) PCT Filed: Apr. 15, 2022
(86) PCT No.: PCT/US2022/024997

§ 371 Date: Oct. 16, 2023

Related U.S. Application Data
(60) Provisional application No. 63/175,423, filed on Apr. 15, 2021.

(51) Int. Cl. A61N 7/00

(52) U.S. Cl. CPC A61N 7/00

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. In particular, provided herein are optimized systems and methods that provide targeted, efficacious histotripsy in a variety of different regions and under a variety of different conditions without causing undesired tissue damage to intervening/non-target tissues or structures.

### PRIORITY CLAIM

This application is related to U.S. Provisional Application No. 63/175,423, titled “DESIGN AND FABRICATION OF THERAPEUTIC ULTRASOUND TRANSDUCER WITH ARBITRARILY SHAPED, DENSELY PACKING, REMOVABLE MODULAR ELEMENTS” and filed on Aug. 15, 2021, which s herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel ultrasound systems configured to treat tissue, including histotripsy which is configured to produce acoustic cavitation, methods, devices and procedures for the minimally and non-invasive treatment of healthy, diseased, and/or injured tissue. The histotripsy systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Many medical conditions require invasive surgical interventions. Invasive procedures often involve incisions, trauma to muscles, nerves and tissues, bleeding, scarring, trauma to organs, pain, need for narcotics during and following procedures, hospital stays, and risks of infection. Non-invasive and minimally invasive procedures are often favored, if available, to avoid or reduce such issues. Unfortunately, non-invasive and minimally invasive procedures may lack the precision, efficacy, or safety required for treatment of many types of diseases and conditions. Enhanced non-invasive and minimally invasive procedures are needed, preferably not requiring ionizing or thermal energy for therapeutic effect.

Histotripsy, or pulsed ultrasound cavitation therapy, is a technology where extremely short, intense bursts of acoustic energy induce controlled cavitation (rapid microbubble formation and collapse) within the focal volume. The vigorous expansion and collapse of these microbubbles mechanically homogenizes cells and tissue structures within the focal volume. This is a very different end-result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, and high-intensity focused ultrasound (HIFU), Histotripsy relies on the mechanical action of cavitation for tissue destruction.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is an example of a transducer array scaffold.

FIG. 3 is an example of an output of a simulation tool that divides the surface of a transducer into multiple concentric rings.

FIG. 4 shows a transducer module mounted within a scaffold.

FIGS. 5A-5B show a CAD rendering of an abdominal transducer array.

FIG. 5C illustrates a method of designing a transducer array.

FIGS. 6A-6B show a rendering of a segmented aperture generated by a simulation tool.

FIG. 7 is a chart which shows electronic focal steering range as a function of element count.

FIG. 8 is a chart which shows the impact of element enclosure thickness on active area.

FIGS. 9A-9B illustrate renderings of an array assembly with an imaging probe retracted (FIG. 9A) and extended (FIG. 9B).

FIGS. 10A-10B show a transducer module which includes a pair of matching layers and a weight positioned on top to apply force along the axis of the module.

FIG. 11 illustrates a completely assembled focused phased array ultrasound transducer with 92% aperture utilization and arbitrarily shaped, replaceable elements.

FIG. 12 shows the transmission through the skullcap at 500 kHz, 700 kHz, 900 kHz, 1 MHz and 2 MHz through sections of excised human skullcaps.

FIGS. 13A-13B show a transcranial transducer array comprising 360 individual square elements with an aperture of 30 cm.

FIG. 14 is another view of a transcranial transducer array.

FIG. 15A shows the spatiotemporal variation through a section of an excised human skullcap highlighting the different “hypothetical element sizes”.

FIG. 15B shows the simulated effects of aberration on a histotripsy waveform for a hypothetical 40×40 mm element.

FIG. 16 shows the average (n=7) fraction of an unaberrated waveform as a function of hypothetical element sizes.

FIGS. 17A-17B illustrate one embodiment of the array modules for the transcranial array application.

FIG. 18 shows the pressure waveform produced from a single module at 150 mm for a peak drive voltage of 3 kV.

FIG. 19A is a chart showing the output of the modules at 150 mm (the focal distance of the array) as a function of drive voltage in the free field and through the human skull.

FIG. 19B is a chart showing the trans-skull transmission of a single module through the skull was plotted as a function of the drive voltage.

FIG. 20 illustrates the peak-negative pressure output of the 360 element hemisphere array transducer simulated across a ±50 mm electronic focal steering range.

### SUMMARY OF THE DISCLOSURE

Histotripsy produces tissue homogenization down to the subcellular scale through dense energetic bubble clouds generated by short, high-pressure, ultrasound pulses. When using pulses shorter than 2 cycles, the generation of these energetic bubble clouds only depends on where the peak negative pressure (P−) exceeds an intrinsic threshold for inducing cavitation in a medium (typically 26-30 MPa in soft tissue with high water content).

A histotripsy transducer array is provided, comprising a scaffold, and a plurality of transducer elements arranged on the scaffold such that the ultrasound transducer array has a packing density greater than or equal to 90%, wherein the array is configured to transmit histotripsy pulses at an amplitude exceeding a cavitation threshold of one or more bubble cloud locations in a target tissue.

In some embodiments, the scaffold is concave. In other embodiments, the scaffold has a generally rectangular shape.

In some implementations, some of the plurality of transducer elements are arranged within a central region of the scaffold in a plurality of concentric rows of transducer elements.

In one example, some of the plurality of transducer elements are arranged in one or more peripheral regions of the scaffold adjacent to the central region.

In some embodiments, the transducer elements in the one or more peripheral regions are arranged in arced rows of transducer elements.

In some examples, the transducer array includes an opening in the scaffold to accommodate an imaging probe disposed within the central region.

In some embodiments, the plurality of transducer elements each have the same surface area. In other embodiments, the plurality of transducer elements have varying surface areas.

In some embodiments, the plurality of transducer elements are separated by a layer of epoxy less than or equal to 125 microns thick.

In one example, the scaffold includes a geometry optimized for transmission of ultrasound waves into a target tissue within the patient's abdomen. In another embodiment, the scaffold includes a geometry optimized for transmission of ultrasound waves into a target tissue within the patient's skull.

In some examples, the scaffold is hemispherical.

A method of designing a transducer array geometry for a target tissue is provided, comprising acquiring a plurality of images of a target tissue, generating an image segmentation mask for each of the plurality of images corresponding to the target tissue, generating a 3D model of the target tissue from the image segmentation masks, projecting rays from a point in the 3D model corresponding to the target tissue to distributed discrete points on a sphere to identify available acoustic windows, summing acoustic windows and excluding regions where less than a threshold value is common across the summed acoustic windows.

In some embodiments, acquiring the plurality of images comprises capturing the plurality of images in real-time.

In other embodiments, acquiring the plurality of images comprises acquiring the plurality of images from a database of historical images.

In one example, the threshold value comprises 80%.

In one implementation, the method further includes segmenting the common aperture into a plurality of nesting arc segment-shaped elements.

In some examples, each of the nesting arc segment-shaped elements have an approximately equal aspect ratio and surface area.

### DETAILED DESCRIPTION

Provided herein are systems and methods that provide efficacious non-invasive and minimally invasive therapeutic, diagnostic and research procedures. In particular, provided herein are optimized systems and methods that provide targeted, efficacious histotripsy in a variety of different regions and under a variety of different conditions without causing undesired tissue damage to intervening/non-target tissues or structures.

Balancing desired tissue destruction in target regions with the avoidance of damage to non-target regions presents a technical challenge. This is particularly the case where time-efficient procedures are desired. Conditions that provide fast, efficacious tissue destruction tend to cause undue heating in non-target tissues. Overheating can be avoided by reducing energy or slower delivery of energy, both of which run contrary to the goals of providing a fast and efficacious destruction of target tissue. Provided herein are a number of technologies that individually and collectively allow for fast, efficacious target treatment without undesired damage to non-target regions.

The system, methods and devices of the disclosure may be used for the minimally or non-invasive acoustic cavitation and treatment of healthy, diseased, and/or injured tissue, including in extracorporeal, percutaneous, endoscopic, laparoscopic, and/or as integrated into a robotically-enabled medical system and procedures. As will be described below, the histotripsy system may include various electrical, mechanical and software sub-systems, including a Cart, Therapy, Integrated Imaging, Robotics, Coupling, and Software. The system also may comprise various Other Components, Ancillaries, and Accessories, including but not limited to patient surfaces, tables or beds, computers, cables and connectors, networking devices, power supplies, displays, drawers/storage, doors, wheels, illumination and lighting, and various simulation and training tools, etc. All systems, methods, and means of creating/controlling/delivering histotripsy are considered to be a part of this disclosure, including new related inventions disclosed herein.

In one embodiment, the histotripsy system is configured as a mobile therapy cart, which further includes a touchscreen display with an integrated control panel with a set of physical controls, a robotic arm, a therapy head positioned on the distal end of the robot, a patient coupling system and software to operate and control the system.

The mobile therapy cart architecture can comprise internal components, housed in a standard rack mount frame, including a histotripsy therapy generator, high voltage power supply, transformer, power distribution, robot controller, computer, router and modem, and an ultrasound imaging engine. The front system interface panel can comprise input/output locations for connectors, including those specifically for two ultrasound imaging probes (handheld and probe coaxially mounted in the therapy transducer), a histotripsy therapy transducer, AC power and circuit breaker switches, network connections and a foot pedal. The rear panel of the cart can comprise air inlet vents to direct airflow to air exhaust vents located in the side, top and bottom panels. The side panels of the cart include a holster and support mechanism for holding the handheld imaging probe. The base of the cart can be comprised of a cast base interfacing with the rack mounted electronics and providing an interface to the side panels and top cover. The base also includes four recessed casters with a single total locking mechanism. The top cover of the therapy cart can comprise the robot arm base and interface, and a circumferential handle that follows the contour of the cart body. The cart can have inner mounting features that allow technician access to cart components through access panels.

The touchscreen display and control panel may include user input features including physical controls in the form of six dials, a space mouse and touchpad, an indicator light bar, and an emergency stop, together configured to control imaging and therapy parameters, and the robot. The touchscreen support arm is configured to allow standing and seated positions, and adjustment of the touchscreen orientation and viewing angle. The support arm further can comprise a system level power button and USB and ethernet connectors.

The robotic arm can be mounted to the mobile therapy cart on arm base of sufficient height to allow reach and ease of use positioning the arm in various drive modes into the patient/procedure work space from set up, through the procedure, and take down. In one embodiment, the robotic arm can comprise six degrees of freedom with six rotating joints, a reach of 850 mm and a maximum payload of 5 kg. The arm may be controlled through the histotripsy system software as well as a 12 inch touchscreen polyscope with a graphical user interface. The robot can comprise force sensing and a tool flange, with force (x, y, z) with a range of 50 N, precision of 3.5 N and accuracy of 4.0 N, and torque (x, y, z) with a range of 10.0 Nm, precision of 0.2 Nm and accuracy of 0.3 Nm. The robot has a pose repeatability of +/−0.03 mm and a typical TCP speed of 1 m/s (39.4 in/s). In one embodiment, the robot control box has multiple I/O ports, including 16 digital in, 16 digital out, 2 analog in, 2 analog out and 4 quadrature digital inputs, and an I/O power supply of 24V/2 A. The control box communication comprises 500 Hz control frequency, Modbus TCP, PROFINET, ethernet/IP and USB 2.0 and 3.0.

The therapy head can comprise one of a select group of various histotripsy therapy transducers and an ultrasound imaging system/probe, coaxially located in the therapy transducer, with an encoded mechanism to rotate said imaging probe independent of the therapy transducer to known positions, and a handle to allow gross and fine positioning of the therapy head, including user inputs for activating the robot (e.g., for free drive positioning). In some examples, the therapy transducers may vary in size (22×17 cm to 28×17 cm), focal lengths from 12-18 cm, number of elements, ranging from 48 to 64 elements, comprised within 12-16 rings, and all with a frequency of 700 kHz. The therapy head subsystem has an interface to the robotic arm includes a quick release mechanism to allow removing and/or changing the therapy head to allow cleaning, replacement and/or selection of an alternative therapy transducer design (e.g., of different number of elements and geometry), and each therapy transducer is electronically keyed for auto-identification in the system software.

The patient coupling system can comprise a six degree of freedom, six joint, mechanical arm, configured with a mounting bracket designed to interface to a surgical/interventional table rail. The arm may have a maximum reach of approximately 850 mm and an average diameter of 50 mm. The distal end of the arm can be configured to interface with an ultrasound medium container, including a frame system and an upper and lower boot. The lower boot is configured to support either a patient contacting film, sealed to patient, or an elastic polymer membrane, both designed to contain ultrasound medium (e.g., degassed water or water mixture), either within the frame and boot and in direct contact with the patient, or within the membrane/boot construct. The lower boot provides, in one example, a top and bottom window of approximately 46 cm×56 cm and 26 cm×20 cm, respectively, for placing the therapy transducer with the ultrasound medium container and localized on the patient's abdomen. The upper boot may be configured to allow the distal end of the robot to interface to the therapy head and/or transducer, and to prevent water leakage/spillage. In preferred embodiments, the upper boot is a sealed system. The frame is also configured, in a sealed system, to allow two-way fluid communication between the ultrasound medium container and an ultrasound medium source (e.g., reservoir or fluidics management system), including, but not limited for filling and draining, as well as air venting for bubble management.

The system software and work-flow can be configured to allow users to control the system through touchscreen display and the physical controls, including but not limited to, ultrasound imaging parameters and therapy parameters. The graphical user interface of the system comprises a work-flow based flow, with the general procedure steps of 1) registering/selecting a patient, 2) planning, comprising imaging the patient (and target location/anatomy) with the freehand imaging probe, and robot assisted imaging with the transducer head for final gross and fine targeting, including contouring the target with a target and margin contour, of which are typically spherical and ellipsoidal in nature, and running a test protocol (e.g., test pulses) including a bubble cloud calibration step, and a series of predetermined locations in the volume to assess cavitation initiation threshold and other patient/target specific parameters (e.g., treatment depth), that together inform a treatment plan accounting for said target's location and acoustic pathway, and any related blockage (e.g., tissue interfaces, bone, etc.) that may require varied levels of drive amplitude to initiate and maintain histotripsy. Said parameters, as measured as a part of the test protocol, comprising calibration and multi-location test pulses, are configured in the system to provide input/feedback for updating bubble cloud location in space as needed/desired (e.g., appropriately calibrated to target cross-hairs), as well as determining/interpolating required amplitudes across all bubble cloud treatment locations in the treatment volume to ensure threshold is achieved throughout the volume. Further, said parameters, including but not limited to depth and drive voltage, may be also used as part of an embedded treatability matrix or look up table to determine if additional cooling is required (e.g., off-time in addition to time allocated to robot motions between treatment pattern movements) to ensure robust cavitation and intervening/collateral thermal effects are managed (e.g., staying below t43 curve for any known or calculated combination of sequence, pattern and pathway, and target depth/blockage). The work-flow and procedure steps associated with these facets of planning, as implemented in the system software may be automated, wherein the robot and controls system are configured to run through the test protocol and locations autonomously, or semi-autonomously. Following planning, the next phase of the procedure work-flow, 3) the treatment phase, is initiated following the user accepting the treatment plan and initiating the system for treatment. Following this command, the system is configured to deliver treatment autonomously, running the treatment protocol, until the prescribed volumetric treatment is complete. The status of the treatment (and location of the bubble cloud) is displayed in real-time, adjacent to various treatment parameters, including, but not limited to, of which may include total treatment time and remaining treatment time, drive voltage, treatment contours (target/margin) and bubble cloud/point locations, current location in treatment pattern (e.g., slice and column), imaging parameters, and other additional contextual data (e.g., optional DICOM data, force torque data from robot, etc.). Following treatment, the user may use the therapy head probe, and subsequently, the freehand ultrasound probe to review and verify treatment, as controlled/viewed through the system user interface. If additional target locations are desired, the user may plan/treat additional targets, or dock the robot to a home position on the cart if no further treatments are planned.

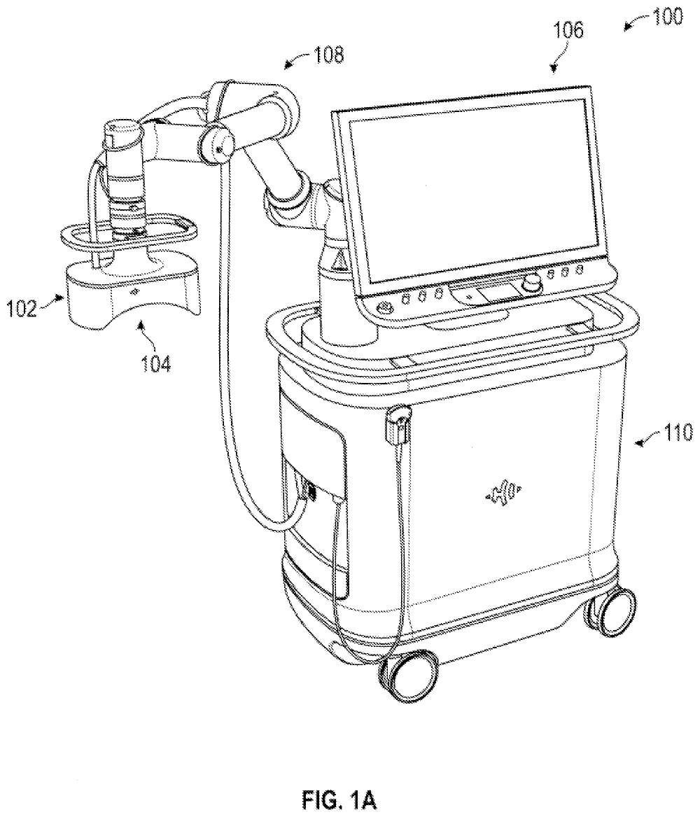

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy).

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with at least a single negative/tensile phase sufficient to cause a cluster of bubble nuclei intrinsic to the medium to undergo inertial cavitation, 2) Shock-Scattering Histotripsy: Delivers typically pulses 3-20 cycles in duration. The amplitude of the rarefactional phases of the pulses is sufficient to cause weak, microscopic bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Transducer Array and Fabrication

A key component of histotripsy therapy is a high-power focused ultrasound transducer array configured to deliver sufficiently high ultrasound pressure and power to generate cavitation in the target tissue. Traditional transducer fabrication techniques include heating a large piece of piezoelectric (PZT) or piezoceramic composite (PCC) material and shaping the material to the appropriate curved shape with high mechanical precision. Next, the shaped PZT or PCC material can be cut into individual transducer elements. Electrode connections are then soldered to the individual transducer elements. In some implementations, a thin curved matching layer can then bonded to the curved PZT or PCC transducer elements. One advantage of the traditional fabrication approach is that the packing density (area occupied by PZT or PCC/total surface area of the array) is relatively high (up to 90%), by leaving small spacing between individual elements. As the ultrasound power output of a transducer array is proportional to the surface area of PZT or PCC, the high packing density maximizes the ultrasound power output. However, PCC needs kerf gaps >0.5 mm between active elements for electric isolation to prevent arcing. This can account for a large fraction of the active area for arrays with small elements, thus the packing density for array with small elements (e.g., <5 mm) can be low (e.g., <60%).

Due to the delicacy of the traditional fabrication process, this method can only be performed in a labor-intensive manner. Device inconsistency due to variability of workers' skill level has also been a large barrier for mass production. The individual traditional elements are permanently embedded within the array and cannot be replaced. For a transducer array with multiple elements (e.g., hundreds or more), if certain elements are broken, either the array needs to be used at a reduced capacity or the entire array needs to be replaced. Given the high requirement of ultrasound power or pressure output histotripsy therapy, an array functioning at partial capability may compromise the ability to treat certain patient populations or target tissues. Additionally, target tissues blocked by bones or gassy organs such as the lung can require an available acoustic window that may not be a typically geometrically symmetric shape suited for traditional fabrication.

This disclosure provides novel ultrasound transducer arrays including methods to design and fabricate an ultrasound array transducer with a high packing density and removable modular elements facilitated by rapid prototyping and arbitrarily shaped modular elements. Such ultrasound transducer arrays can be used for histotripsy and other therapeutic ultrasound applications. The pre-designed arbitrary shaped elements can be made with rapid prototyping to maximally utilize the transducer surface area. Each of these individual elements can be built into a removable element by cutting a ceramic material to the pre-designed shapes, bonding the elements to 3D printed backing and matching layers, and then coating the elements in a thin high dielectric strength film. This coating is configured to insulate each element, while minimize the spacing between removable element modules. The element modules can then be assembled to a transducer scaffold.

The ultrasound transducer arrays of the present disclosure provide the following benefits and advantages over traditional transducer arrays: 1) High packing density—the techniques, systems, and methods described herein provide transducer arrays with a high packing density (e.g., area occupied by PZT or PCC/total array surface area >90%). In comparison, traditional transducer arrays typically have a packing density below 70%, 2) Transducer element shape—the techniques, systems, and methods described herein provide the use of multiple pre-designed arbitrary shapes of elements to maximally utilize the transducer surface area. For example, the surface area of the transducer array can be divided into multiple concentric rings, and each ring can then split into trapezoidal shaped elements of equal area, while the trapezoidal elements at different rings may be of different sizes, 3) Array geometry—the techniques, systems, and methods described herein provide customized array geometries based on the available acoustic window for a given target tissue without blockage from bones or gassy organs. The electronic steering range and the aberration of acoustic propagation through the acoustic path for a given target tissue can further be used to determine the individual transducer element sizes, 4) Fabrication—the techniques, systems, and methods described herein provide a novel way of enclosing and electrically insulating transducer elements of the transducer array. For example, the PZT or PCC individual transducer elements can be bonded to 3D printed backing-mounts and front matching layers of the same shape, such that the profiles of the stacking and bonding element-components are flush. Then the element stack can be coated by a thin layer of epoxy (e.g., 125-microns thick) with high dielectric strength (e.g., 1-2 kV per 25 microns at 75-microns thickness) to serve as a very thin electrical insulator. Without depending on a modular housing wall to isolate each element, the spacing between elements can then be significantly reduced, and the packing density of the array can be increased.

The present disclosure provides simulation algorithms to enable selection of the optimized frequency, array geometry, and element geometry to minimize aberration, maximize focal pressure, and achieving a sufficiently large electric steering range to maximize the treatment speed. In one implementation, the simulation tool is built on a large database of historical patient imaging, such as patient MRI/CT scans. The simulation tool can further utilize previous transducer data, material data, and tissue testing data.

### Transducer Array Design, Components, and Manufacture

Ultrasound transducer arrays of the present disclosure are generally customized based on the target tissue to be treated, including a customized array geometry, an optimized operation frequency, customized transducer element size and shape, individually replaceable transducer element modules, and an array scaffold configured to hold the transducer element modules.

### Center Frequency

Considerations for selecting the center frequency of a customized transducer array can include frequency-dependent parameters such as focal-zone dimensions, electronic focal steering range, soft-tissue attenuation, incidental and intrinsic cavitation thresholds, focal gain, pressure output, and nonlinear acoustic propagation. Using a low center frequency for histotripsy therapy has several advantages. For example, the full width half maximum (FWHM) of the achievable electric focal steering (EFS) pressure-profile scales roughly linearly with the reciprocal of frequency. Acoustic signals at lower frequencies experience less attenuation which can be seen in the power law of the attenuation coefficient,

α=α_0f\hat{ }n

where α is expressed in units of dB/cm, n ranges from 1 to 2 for soft tissues, and f is the center frequency expressed in units of MHz. The EFS range also increases with decreasing frequency.

Using a low center frequency also has several potential disadvantages. In the absence of attenuation, pressure output scales linearly with frequency and focal gain is also greater at higher frequencies due to a more confined focal zone, making higher frequencies desirable for small transducers and indications in superficial tissues. A lower frequency also results in larger focal zone and lower treatment precision. For deep histotripsy targets like the liver, however, the selection of an optimal center frequency can be distilled largely to the opposing considerations of focal pressure and attenuation.

In some embodiments, individual transducer elements can be made with different frequencies to build a frequency compounding array.

### Array Geometry

To design the array for treatment of a specific target tissue location, a simulation algorithm configured to analyze a historical database of patient imaging (such as ultrasound, CT/MRI images) can be used to segment tissue structures and generate image segmentation masks for each image corresponding to the target tissue, such as liver, pancreas, skin, bone, lung, bowel, stomach, etc. The simulation algorithm can then be configured to generate a 3D model of the segmented tissue (e.g., from the image segmentation masks). In one implementation, the simulation algorithm is configured to project rays from a point in the target tissue to distributed discrete points on a sphere with a center at the target point and a radius equal to the transducer arrays proposed focal length. Each ray can be assigned a binary value (blocked or unblocked). For example, a “blocked” status can be assigned to those rays that fell outside the scan region (by intersecting structures within the superior-most or inferior-most 2D images), intersected overlying bony or gassy tissue structures (lung, stomach, bowel, bone, etc.), or fell on an intracorporeal portion of the sphere. An “unblocked” status can be assigned to the remaining rays within the scan region.

With the available acoustic windows for each target tissue identified, a “common aperture” can be established by 1) aligning all target points and rotating the acoustic windows with respect to each other to achieve maximum overlap, 2) summing the acoustic windows to generate a “heat map” showing the variation in overlap on the summed aperture surface, and 3) setting a threshold to exclude regions where, for example, less than 80% of the aperture is common across all cases analyzed. Based on the results of this analysis, the algorithm can select a spherical section defined by a truncated circular aperture-profile.

### Scaffold

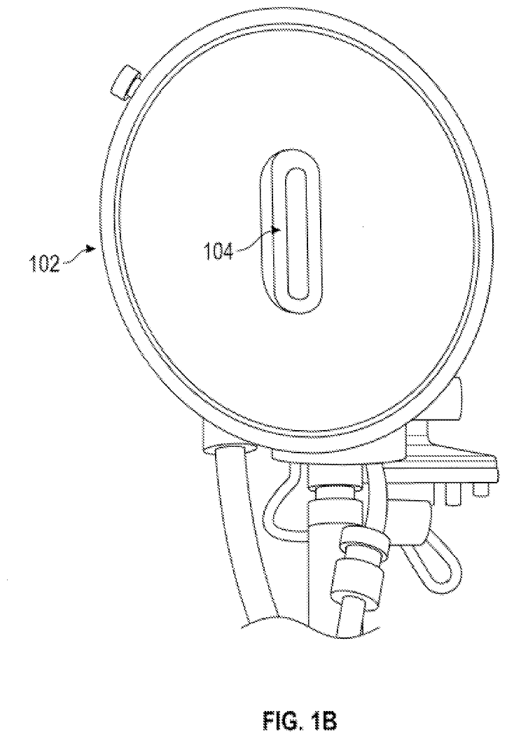

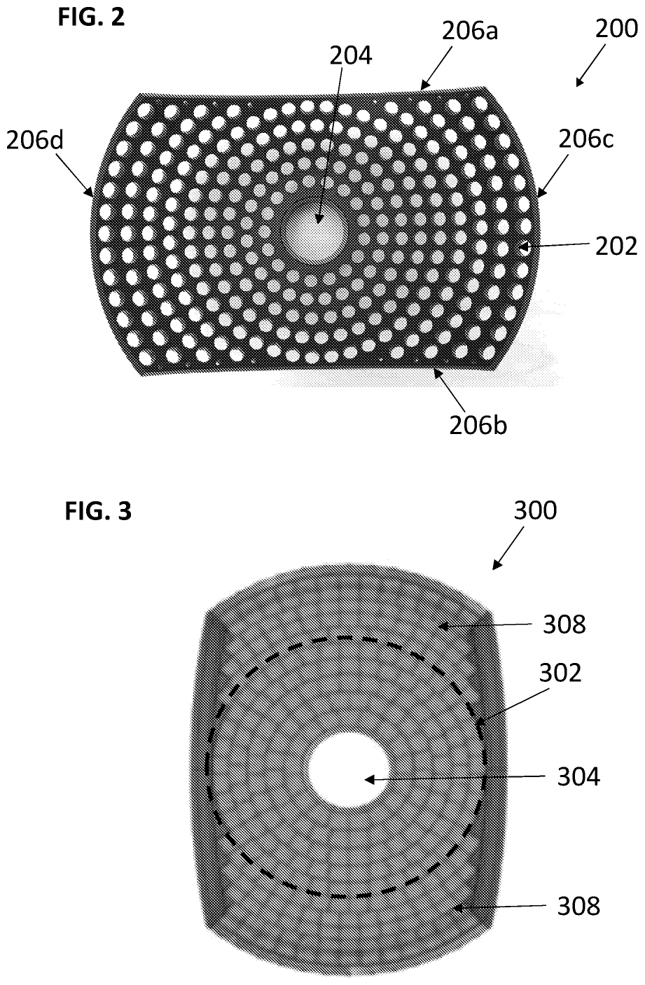

The transducer arrays described herein generally include an array scaffold or array shell, which is a supporting member with a specifically designed size and shape configured to hold all of the transducer elements of the transducer array in the proper position and orientation. An array scaffold or shell of any specific shape or size can be designed and fabricated using rapid prototyping, 3D printing, or traditional machining. Individual transducer elements can then be assembled/affixed to the scaffold. The transducer array scaffold can be concave, convex, flat, or any other customized shape. In some implementations, the scaffold contains mechanical locking structures to secure the individual elements and also allow individual elements to be removed and replaced easily. One example of a transducer array scaffold 200 is shown in FIG. 2. As shown, the scaffold 200 can include a plurality of openings 202 configured to receive individual transducer elements or transducer modules, which will be described below. The scaffold further includes an optional imaging probe opening 204 configured to receive an ultrasound imaging probe for imaging the therapy and/or target tissue in real-time.

The scaffold can be designed and built for any specific target tissue site, to fit any shape or size of the available acoustic window without blockage by bones or other tissue structures like the lungs. While the traditional shape of a transducer array is circular, the scaffold of the present disclosure can be built with rapid prototyping or 3D printing in any arbitrary irregular shape to match the desired acoustic window. Referring to FIG. 2, the transducer array scaffold can comprise a generally rectangular shape. However in this embodiment, sides 206a and 206b can be generally straight, and sides 206c and 206d can be curved or arced. Similarly, the scaffold or shell of array 300 in FIG. 3 has a similar shape, but all four sides can be curved or arced.

The array scaffold can be designed to incorporate components other than the transducer element modules. For example, a larger hole can be incorporated into the array scaffold to allow an ultrasound imaging probe. Spaces can also be incorporated into the array scaffold to allow optical windows for optical lighting, cameras, hydrophones, or other surgical instruments.

### Element Size/Shape

The transducer arrays described herein require careful pre-design of individual transducer element shape and sizes. A simulation tool/algorithm as described above can be configured to obtain the pre-designed arbitrary shapes and/or arrangement of elements for a given array transducer aperture and target tissue site. For example, referring to FIGS. 2-3, the simulation tool can be configured to provide a transducer array (200, 300) having a concentric region 302 made up of multiple concentric rings of transducer elements positioned in a central region of the transducer array. As shown in FIGS. 2-3, the concentric region can comprise several concentric rows or rings or transducer elements. In some examples, the concentric rings can surround imaging probe opening 204/304, as shown. In other examples without an imaging probe opening, the concentric rings can fill in the entire central region 302. The transducer elements of the FIG. 2 embodiment are generally circular transducer elements, whereas the transducer elements of FIG. 3 are generally trapezoidal transducer elements. In some embodiments, the transducer elements in different concentric rings may have different sizes. In one implementation, the size of all transducer elements can be the same or similar sizes, such that the electric impedance of all elements is identical or similar, allowing the use of the same driving electronics and amplifier for all transducer elements.

In the embodiments of FIGS. 2-3, the transducer arrays can further include transducer elements arranged in peripheral regions adjacent to the central region (e.g., regions 308) that include rows of transducer elements but not full concentric rings of elements. As shown in FIG. 3, the regions 308 can include arced or curved rows of transducer elements that extend outwards to fill out the remaining surface area of the transducer array shell. The configurations described above and herein allow for a high packing density of transducer elements within the total surface area of the array scaffold or shell. In contrast to prior transducer designs which achieve a packing density on the order of only 70% or less, the transducer arrays described herein can provide a packing density of greater than 90%.

Individual transducer elements can be flat piezoelectric crystals, while the focusing of the transducer array is achieved with the transducer scaffold geometry. Flat piezoelectric crystals are of low cost and widely available from a number of suppliers. Individual elements can also be curved, focused elements.

### Modules

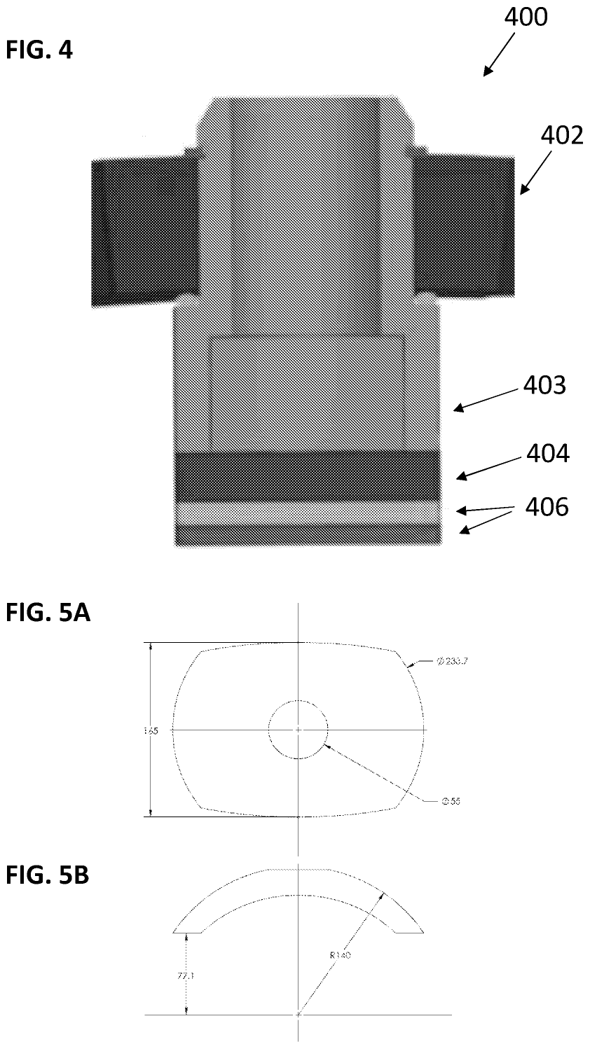

Transducer elements can be mounted/affixed to individual transducer modules, which can then be inserted/affixed to the scaffold of the transducer array. As shown in FIG. 4, a transducer module 400 can be mounted within a scaffold 402 (and also such as scaffolds 200 and 300 in FIGS. 2-3). The module can include, for example, a backing material 403, a transducer element 404, and one or more matching layers 406. Individual modules are removable and replaceable. Individual modules can be performance tested before assembly into the final transducer, improving the yield rate and further reducing the production cost of the array. Damaged or worn array modules can be replaced individually, reducing maintenance costs of the array. All transducer element modules can be routinely calibrated, such that sub-performance elements modules can be replaced to ensure high performance of the system as a whole. The individual elements can be built in large quantities and at low cost to provide a stock of readily available replacement elements should they be needed. As such, replacing element modules would be fast and cheap.

Each element module is connected to individual driving electronics to enable electronic focal steering, aberration correction, and other array functions.

### Electrically Insulating Coating

As described above, one or more acoustic matching layer can be bonded to the front of the piezoelectric material to match the active element. For traditional transducers, matching layers must be formed from very fragile, thin shells which are more difficult and expensive to manufacture and bond to the active layer.

### Manufacturing Methods

The new fabrication process enables a high-packing density and high-power output of the ultrasound array transducer, making it practical for therapeutic ultrasound use.

This approach is amenable to mass production under quality control processes with the potential for substantial automation of most of the production. For example, module backings and scaffold could be mass produced through injection molding at very low cost. The aligned stackup of materials for bonding is suitable for automated assembly.

This process is conducive to a wider range of piezoelectric materials (such as those with low Curie temperature) as the assembly can be performed with or without applying mechanical or thermal stress to the material.

### Abdominal Region Transducer Array

One specific implementation described herein is an ultrasound array transducer configured for treatment of target tissue sites in an abdominal region of a patient.

### Center Frequency

Selection of the Array's Center Frequency for treatment within the abdominal region: as described above, considerations for selecting the center frequency of the array can include frequency-dependent parameters such as focal-zone dimensions, electronic focal steering range, soft-tissue attenuation, the incidental and intrinsic cavitation thresholds, focal gain, pressure output, and nonlinear acoustic propagation. For deep histotripsy targets like the liver, however, the selection of an optimal center frequency can be distilled largely to the opposing considerations of focal pressure and attenuation. To guide the selection of a center frequency for this abdominal array, a series of tests were performed using test-elements at 0.5, 0.7, 0.9, 1, and 2 MHz together with known aberrators, and 700 kHz was selected as the center frequency to constructed a liver array transducer to ensure a low attenuation and sufficiently large electronic steering range in the following example, however, the frequency in the range of 0.5-1.0 MHz can be used.

### Array Geometry

To design the array for treatment of the abdominal region, a series of images of a target tissue region can be taken or acquired from a historical database. For example, historical images of abdominal treatment patients can be acquired and analyzed by a simulation tool/algorithm to segment tissue structures and generate masks for each 2D image corresponding to the target tissue (e.g., liver, pancreas, skin, bone, lung, bowel, and stomach). The images can comprise any imaging modality known in the art, including ultrasound, CT, MRI, etc. A 3D model of the segmented tissue can then be generated by the simulation tool/algorithm. As described above, the simulation tool can be configured to analyze the data by projecting rays from a target point in the target tissue (e.g., an organ or target in the abdominal region) to distributed discrete points on a sphere with a center at the target point and a radius equal to the transducer's proposed focal length. Each ray can then be assigned a binary value (blocked or unblocked).

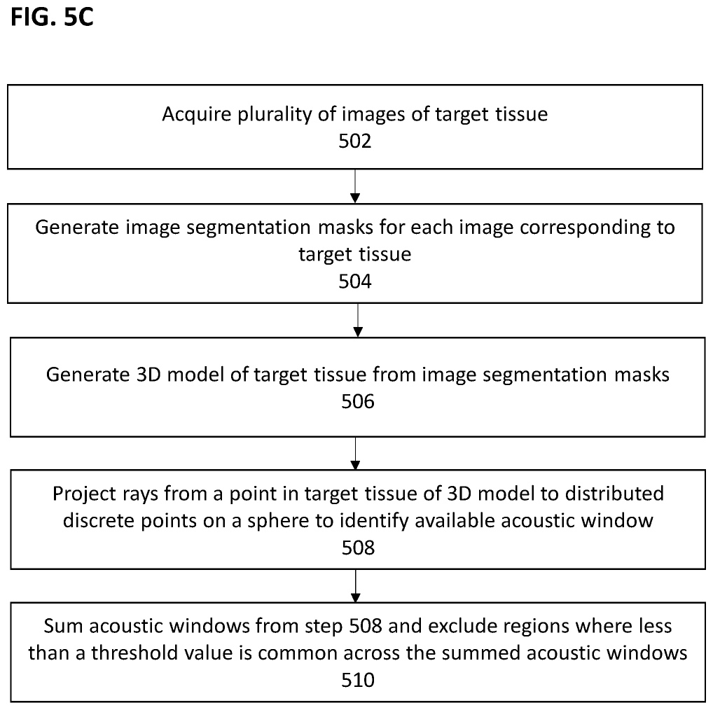

With the available acoustic windows for each patient identified, a “common aperture” can be established by 1) aligning all target points and rotating the acoustic windows with respect to each other to achieve maximum overlap, 2) summing the acoustic windows to generate a “heat map” showing the variation in overlap on the summed aperture surface, and 3) setting a threshold to exclude regions where (e.g., less than 80%) of the aperture was common across all cases analyzed. In one specific implementation, a spherical section is defined by a truncated circular aperture-profile measuring 234 mm in diameter along the widest dimension and 165 mm along the orthogonal dimension was selected, with a focal length of 140 mm. A hole measuring 55 mm can also be established in the center of the aperture to accommodate a standard commercial curvilinear ultrasound imaging probe. A CAD rendering of one example of an abdominal transducer array is shown in FIGS. 5A-5B.

A flowchart of a method of designing a transducer array geometry for a target tissue is shown in FIG. 5C, and can include the steps of:

At step 502 of the flowchart, the method can include, acquiring a plurality of images of a target tissue. The target tissue can comprise, for example, a liver, pancreas, skin, bone, lung, bowel, stomach, or any other organ or target tissue in a human body, etc. In some examples, the images can be acquired in real time with a medical imaging modality, such as ultrasound, CT, MRI, or the like. In other embodiments, historical images of the target tissue can be acquired from a database.

At step 504 of the flowchart, the method can include generating image segmentation masks for each image corresponding to the target tissue. At step 506, the method can include generating a 3D model of the target tissue from the image segmentation masks. In some embodiments, the generating image segmentation masks step and generating a 3D model step of the method can be implemented and executed within a CPU processor or computing system running an algorithm, such as a simulation algorithm or software.

At step 508 of the flowchart, the method can include projecting rays from a point in the target tissue of the 3D model to distributed discrete points on a sphere with a center at the target point and a radius equal to the transducer arrays proposed focal length. Each ray can be assigned a binary value (blocked or unblocked). For example, a “blocked” status can be assigned to those rays that fell outside the scan region (by intersecting structures within the superior-most or inferior-most 2D images), intersected overlying bony or gassy tissue structures (lung, stomach, bowel, bone, etc.), or fell on an intracorporeal portion of the sphere. An “unblocked” status can be assigned to the remaining rays within the scan region to identify the available acoustic window.

At step 510 of the flowchart, the method can include identifying a common aperture that includes summing the acoustic windows from step 508 and excluding regions where less than a threshold value is common across the summed acoustic windows. In some examples, this step can include 1) aligning all target points and rotating the acoustic windows with respect to each other to achieve maximum overlap, 2) summing the acoustic windows to generate a “heat map” showing the variation in overlap on the summed aperture surface, and 3) setting a threshold to exclude regions where less than a threshold value (e.g., less than 80%) of the aperture is common across all cases analyzed.

### Element Size/Shape

Using the array geometry defined above and shown in FIGS. 5A-5B, the electronic focal steering range can be simulated as a function of element-count with the simulation tool. The simulation tool can be configured to segment the aperture into nesting arc segment-shaped elements with approximately equal aspect ratio and surface area. Arc segments can be generated by dividing the aperture into concentric rings and then further dividing each ring in the azimuthal direction. Having roughly square (vs. elongated rectangular, for example) generally improves the ability to perform localization through the reception of acoustic signals. Having roughly equal surface area for each element simplifies the construction of the array's electronic driving circuitry. Triangular regions along the array's flanks can be left unpopulated for these reasons and due to the high additional per-element cost associated with making eight additional shapes for which there would be only 16 elements.

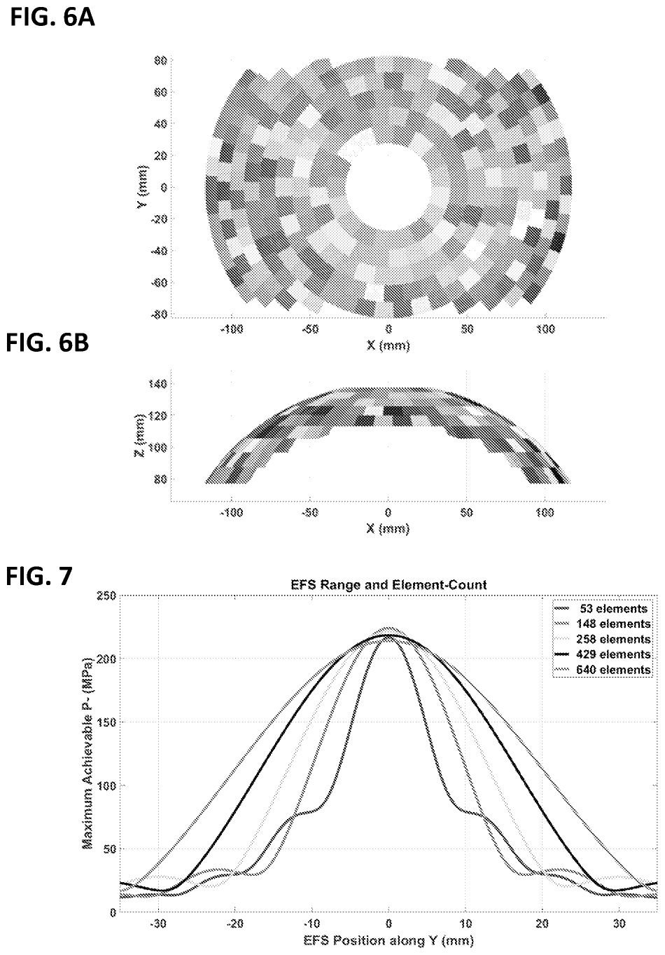

The simulation tool can be configured to loop through a series of targeted element-counts ranging from 25 to 750 in increments of 25 elements. In order to maintain the input geometric constraints, the actual element-count can be allowed to deviate from the targeted count by approximately 5%. If a precise number of elements is desired, the basic aperture dimensions can be modified slightly to accommodate this requirement. After each iteration, the simulation tool can be configured to save a data file containing the 3D information fully defining each element (center position, profile, area etc.). A rendering of the segmented aperture generated by the simulation tool appears in FIGS. 6A-6B.

The simulation tool can also be configured to evaluate the electronic focal steering (EFS) range of the apertures of the array above. For each aperture-configuration, the contribution of a perimeter around each element (e.g., measuring 250 microns) necessary for electrical insulation and mechanical clearance can be subtracted from the total area. Selected results of this analysis are displayed in FIG. 7, which shows electronic focal steering range as a function of element count. Referring still to FIG. 7, a maximum achievable P− is estimated based on linear summation. The simulation tool assumes a gap between elements' active piezoelectric material of 0.5 mm. Utilizing the full aperture (in the absence of any gaps or unpopulated regions at the aperture's flanks), the maximum P− at the focus can be estimated to be 246 MPa. Packing-density is modest for low element-counts (due to the unpopulated triangular regions on the array's flanks), optimal near 150 elements, and begins to diminish thereafter at higher element-counts (due to the increased area occupied by inter-element gaps).

This analysis revealed that an array with 384 elements, each with an area of 99.4+/−7.2 mm², would have an estimated FWHM EFS range of 32 mm in the least-steerable direction. At this EFS position, 16 mm off axis in the y-direction, the proposed array-configuration was estimated to generate 111 MPa in the free field. The full width of the EFS range in the y-direction at 26 MPa (approximate cavitation intrinsic threshold in the free field (14)) and 52 MPa (approximate de-rated threshold in vivo) were estimated to be 50 mm and 44 mm, respectively. An aperture with 384 approximately 1-cm² elements was used as the starting point for the mechanical design.

### Modules

Each transducer element may be affixed to the array scaffold by mating threads on the interior of the hole or secured on the backside of the scaffold by a nut modified with by the addition of a slot to form a C-shape which allows the cable to pass through it. A threaded mounting hole requires the element to be rotated which inherently constrains the packing density of the aperture. The C-nut design allows more design flexibility but nevertheless requires significant spacing between elements to allow clearance for the nuts and a tightening tool to rotate.

In some implementations, the mechanical design of modular elements for this array is held fast within the scaffold by a removable low-profile stainless steel retaining ring mated with a radial groove on the shaft of the module assembly. An O-ring can then be fitted between the module and the scaffold on the concave side to provide a small spring force (approximately 7 N). This allows the module to be rigidly fixed during use and absorbed slight dimensional deviations from parts' nominal values, easing installation. Other embodiments may include modules secured by various other means including various threaded fasteners, temporary or permanent adhesives, and/or other means of attachment.

The diameter of the through-holes in the scaffold are dictated by the electrical connector which passes through it and the size of the element which it retains. In turn, the diameter of the hole determines the minimum spacing and size of elements. It is therefore desirable to use the smallest connector which meets other practical considerations. Other embodiments may include designs which route cables along the interior or exterior of the scaffold. Connectors may be joined to the module wires before or after mating modules to the scaffold.

In one implementation, the size of the selected connector and corresponding scaffold through-hole drove element-size to a minimum value of 11.4 mm, which provides a tight fit for the connector, O-ring, and adjacent elements. To relax clearances and provide easier manufacturability and fit, the element size can be set to, for example, 12 mm. With this element size, an array configuration can be achieved with 258 elements, a mean area of 146.7 mm²+/−6.2 mm², and an EFS range of 26.2 mm and 36 mm at FWHM and 52 MPa, respectively. Relative to the area of the full aperture, this design achieves 88.4% active area. Relative to the area of the full aperture minus the area of triangular regions at the aperture's flanks, this design achieves 92.1% active area. The triangular regions (which constitute approximately 4% of the total area of the aperture) could be easily populated with active material if desired.

### Electrically Insulating Coating

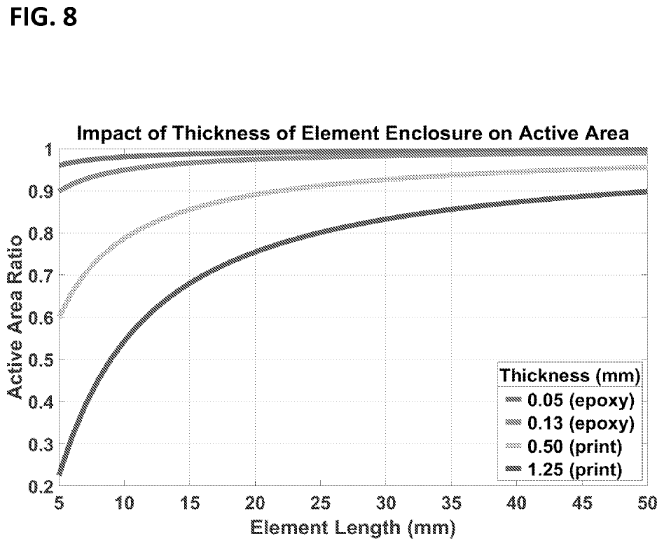

The present disclosure further provides a novel technique for enclosing and electrically insulating each transducer element by applying a thin (e.g., less than or equal to 125-microns thick) layer of epoxy with exceptionally high dielectric strength (e.g., 1-2 kV per 25 microns at 75-microns thickness) to serve as a very thin electrical insulator. In this manner, the gap between active piezoelectric components can be greatly diminished, resulting in substantially higher active area for the acoustically emissive surface of the element. For example, if a square element of total length L on a side and an enclosure thickness of T are assumed, the ratio of active area to total area is described by,

$$R = \frac{{( L - 2 T )}^{2}}{{L}^{2}}$$

where the term within the brackets represents the length of the piezoelectric material. In the case of a 3D printed housing, the term T must also include a small mechanical clearance between the housing and the crystal. As displayed in FIG. 8, the relative loss of active area is marginal for large elements but substantial for small ones. Assuming a 12-mm square element and a nominal 0.13-mm clearance between the 3D printed housing and the piezoceramic, the active area is 17-61% greater using epoxy-coating for insulation rather than a 3D printed housing.

FIG. 7 shows a comparison of techniques for providing an electrically insulating enclosure for elements square elements of various sizes. For small elements, a 3D printed enclosure occupies a substantial portion of the acoustically emitting surface of the element. By instead stacking and bonding element-components such that their profiles are flush, then coating this assembly in a thin layer of epoxy for electrical insulation, the ratio of active area to total area of the acoustically emissive surface can be greatly improved.

### Manufacturing Methods

In one specific implementation, the transducer's scaffold, which serves as a rigid mount for elements, can be machined from 6061-T-6 aluminum and type 3 hard anodized per MIL-A8625 with a minimum thickness of 0.05 mm to provide corrosion resistance. The scaffold is configured to incorporate a cylindrical projection on the backside which has a bore 50 mm in diameter and a bolt pattern at the end of the bore. This feature can be used to mount the transducer to a positioning system and house the mechanism which controls the ultrasound imaging probe. The ultrasound imaging probe can be secured within a 3D printed clamshell-style housing which can then be fastened to a hollow actuation shaft machined from acetal copolymer. The actuation shaft is sized to fit within the bore of the scaffold and can be precisely rotated by a modified optical rotation stage. The actuation shaft can also be extended up to 71 mm from its retracted position near the therapy elements.

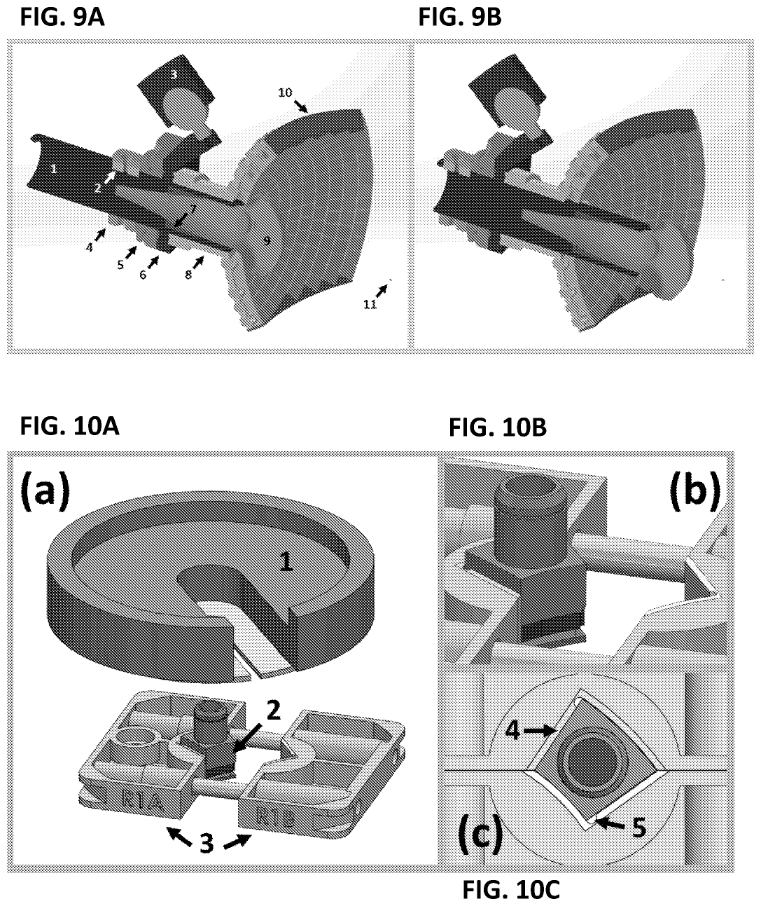

FIGS. 9A-9B illustrate renderings of an array assembly with an imaging probe retracted (FIG. 9A) and extended (FIG. 9B). The array includes an actuation shaft 1, a parallel key with taped hole 2, a ball head mount 3, an actuation shaft mount 4, an optical rotation stage 5, a mounting bracket 6, a 3D printed clamshell mount 7 for the imaging probe, a scaffold 8, an imaging probe 9, a 3D printed trim piece 10, and a geometric focus 11 of the transducer.

Piezoceramic bulk materials are typically manufactured by hydraulically pressing a granular mix of piezoelectric and an organic binder material into a mold. Basic geometric shapes like disks and plates as well as slightly more complex shapes like cylinders and hemispheres are commonly formed this way. The “green” material is then sintered at high temperature to form a crystalline structure of PZT and to evacuate the binder material. Once cool, elements can be trimmed and finished by machining with diamond coated tooling. As a final step, electrodes are applied and the material is poled above the Curie temperature. For large-volume production of the arc segments described herein, elements can be pressed into slightly oversized shapes and then machined or ground down to achieve appropriate tolerances.

Starting from a larger sheet of material, a dicing saw is commonly used to form piezoceramics into rectilinear shapes. More complex two-dimensional shapes can be formed by CNC milling operations but require specialized tooling and elevate the risk of locally de-poling the material by frictional heating. Waterjet cutting presents two key advantages for shaping piezoelectric materials. First, arbitrary and even highly intricate shapes can be cut from delicate materials like ceramic or glass due to the very small and localized machining force. Second, because waterjet cutting does not elevate the temperature of the work-piece, there is no risk of thermal damage. Waterjet machining can comfortably hold standard machining tolerances (+/−0.13 mm) and a few can hold as good as +0.00/−0.05 mm.

The two matching layers, piezoceramic, and element-mount share a common profile and stack flush with each other, therefore requiring a fixture for alignment during assembly. Assembly fixtures can include a vise-like clamp with reliefs that conform to the element's profile and apply pressure to the four lateral sides of the stack. A weight can be positioned on top of the stack to apply force along the axis of the module. An illustration of this system is displayed in FIGS. 10A-10C, which shows a weight 1 above a stack of element components 2, which are positioned within an open assembly clamp 3.

Assembly clamps can be 3D printed in a single operation using a combination of rigid acrylic polymer and elastomeric material. The clamps can include relief features with a profile that closely matches that of the transducer element module, and produces a slight interference fit (approximately 0.06 mm per side) when the jaws of the assembly clamp are closed. The main body of each side of the clamp can be made from rigid material while the inset profile of the relief feature can be lined with a 1-mm layer of elastomeric material designed to conform to the profile of the element-stack. The corners of the inset profile can feature small cutouts which served as vents for excess epoxy. Because the arrays described herein can contained multiple unique arc-segment shapes, each shaped element requires a unique assembly clamp.

The two matching layers, the piezoelectric material, and the backing-mount can be joined using an epoxy. Surfaces of the assembly fixture which come into contact with epoxy can be covered in adhesive-backed PTFE tape and coated with an oil or spray prior to use. Dimensions of the clamp and the desired level of mechanical interference can be specified to account for the thickness of the PTFE tape. Following the application of epoxy to components to be joined and insertion into the clamp, the backing-mount can be filled with epoxy and a custom strain relief mounted on the cable can be inserted into the bore of the backing-mount. A weight as described above can be placed on the entire assembly. The epoxy can then be allowed to cure for at least 24 hours before removing the element module from the clamp assembly. The element module can then be trimmed and lightly sanded using 600-grit sandpaper to remove any excess epoxy which may have accumulated on the exterior surfaces. Elements can then be cleaned and coated with a 125-microns thick layer of epoxy with high dielectric strength epoxy.

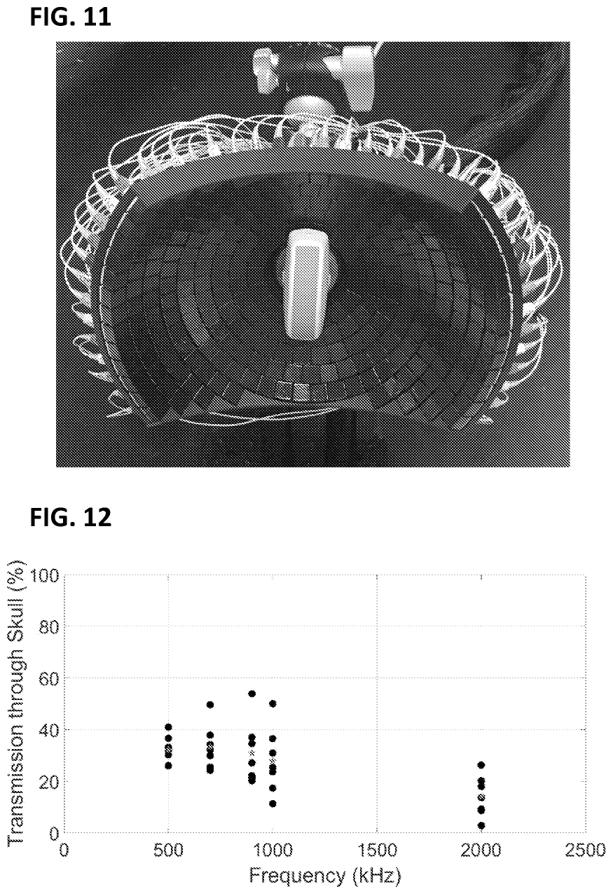

FIG. 11 illustrates a completely assembled focused phased array ultrasound transducer with 92% aperture utilization and arbitrarily shaped, replaceable elements, as described above.

### Transcraneal Transducer Array

Another specific implementation described herein is an ultrasound array transducer configured for transcranial treatment of target tissue sites within the skull of a patient.

### Center Frequency

To determine the optimal center frequency for a transcranial transducer array, the transmission through excised human skullcaps (n=7) at discrete frequencies (500 kHz, 700 kHz, 900 kHz, 1 MHz and 2 MHz) was measured. A degassed, excised human skullcap was placed approximately halfway between a measurement hydrophone and the ultrasound transducer. This distance is similar to the anticipated distanced between the external skull surface and the surface of the array elements in a therapy setting. The skullcap was oriented such that its surface was approximately normal to the ray emitted from the ultrasound transducer. Transducers were electronically driven to produce ˜1.5 cycle acoustic pulses at their center frequency. At each frequency, measurements of the peak-negative pressure were made with and without the skull in place via the capsule hydrophone.

FIG. 12 shows the transmission through the skullcap at 500 kHz, 700 kHz, 900 kHz, 1 MHz and 2 MHz through sections of excised human skullcaps. The mean transmission remained relatively flat between 500 kHz and 1 MHz (roughly 30%). There was a clear decrease in the mean transmission between 1 MHz and 2 MHz. 700 kHz was selected as an optimal center frequency for transcranial transmission to ensure a low attenuation and sufficiently large electronic steering range through the skull.

### Array Geometry

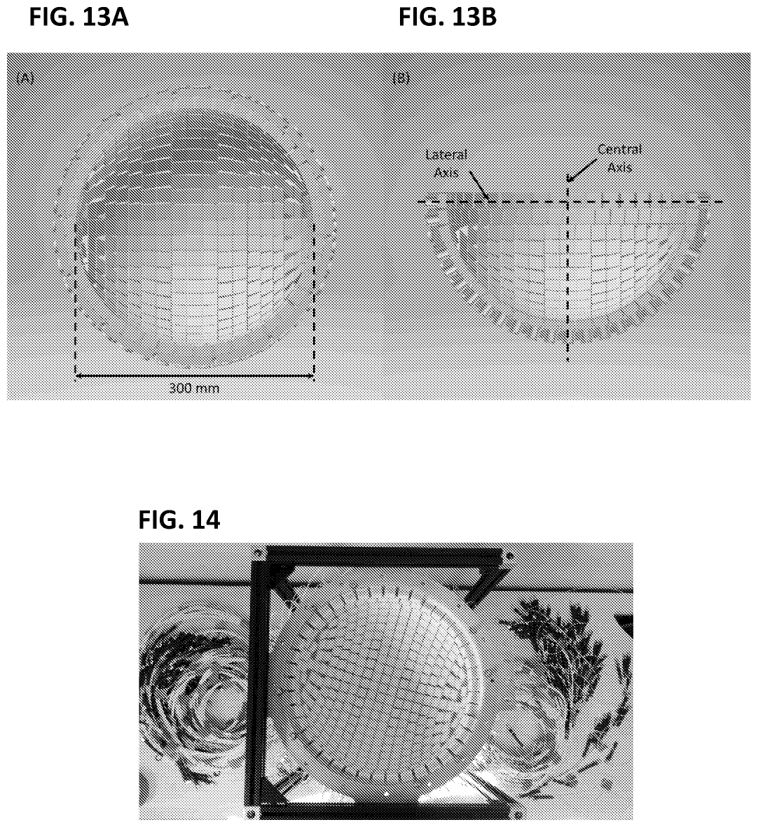

For the transcranial application, a hemispherical array transducer can comprise 360 individual square elements with an aperture of 30 cm, as shown in FIGS. 13A-13B. The elements can be patterned to optimize their packing within the 30 cm diameter hemisphere scaffold. The elements positioned toward the back of the hemisphere scaffold may be circularly patterned around a lateral axis while the two rings of elements positioned near the edge of the hemisphere scaffold may be circularly patterned around a central axis. This packing strategy can be adopted to keep regions of densely packed elements in regions that were thought to follow the natural curvature of the skull. A picture of the completed 360-element, 700 kHz transcranial array is shown in FIG. 14. Overall, the packing strategy allowed the active area of individual transducer modules to occupy 70-90% of the total hemisphere surface area.

### Element Size/Shape

Experiments were also performed to quantify the spatiotemporal variation inherent to the skull and investigate how it may be used to guide considerations for the size of the transducer elements. A degassed, excised human skullcap was placed 30 cm from an ultrasound transducer that was fabricated in-house. This distance was used so that the section of wave propagating through the skull approximated a plane wave. A capsule hydrophone was placed on the other side of the skullcap, approximately 5 mm from the internal surface of the skull. The hydrophone was then scanned through a 40×40 mm grid in the lateral-elevational plane, with a step size of 2 mm. At each location, the acoustic waveform was captured and the time-of-flight (TOF) was measured. TOF measurements were made using the edge of the acquired waveform envelope, which was defined as 15% of the envelope peak. Identical measurements were made without the skullcap and used to calibrate out any temporal variations not caused by propagation through the skull.

The grid of spatially varying time values measured through sections of skullcaps (n=7) was used to simulate the effects of aberration on ˜1.5 cycle waveforms emitted through the skull. This was done by first windowing the 40×40 mm grid into a 40×40, 20×20, 16×16, 12×12, 8×8 and 4×4 mm grid. A Gaussian pulse was then generated in MATLAB to resemble a 700 kHz 1.5 cycle waveform, similar to pulses used in intrinsic threshold histotripsy. To simulate the aberrated waveform for hypothetical element sizes, this un-aberrated waveform was shifted according the relative delays of the pixels within each window size. The shifted waveforms were then summed. The peak-negative amplitude of the shifted, summed waveforms was then divided by the peak-negative amplitude of the un-shifted, summed waveforms. The fraction of the un-aberrated waveform at each hypothetical element size was compared to obtained from the 4×4 mm grid via pair-wise, independent 2-tailed ¬t-tests (α=0.05).

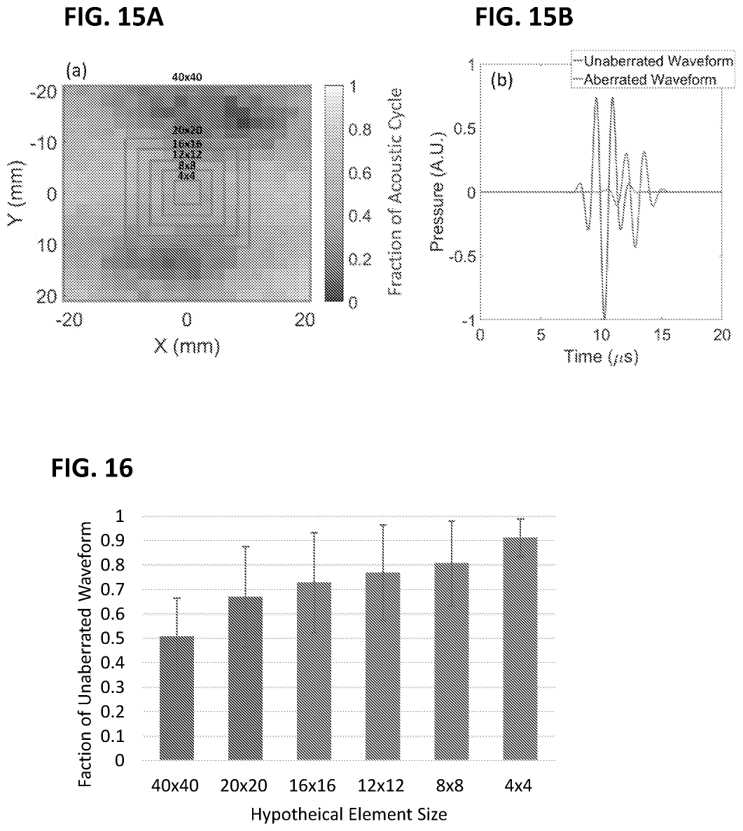

FIG. 15A shows the spatiotemporal variation through a section of an excised human skullcap highlighting the different “hypothetical element sizes” (i.e., window sizes) investigated. FIG. 15B shows the simulated effects of aberration on a histotripsy waveform for a hypothetical 40×40 mm element. The simulated aberrated waveform has a reduced peak-negative pressure in comparison to the unaberrated waveform. The ratio of the peak-negative pressure of the aberrated waveform to the peak-negative pressure of an un-aberrated waveform was calculated to produce the fraction of the un-aberrated waveform.

FIG. 16 shows the average (n=7) fraction of an unaberrated waveform as a function of hypothetical element sizes. The fraction of unaberrated waveform was small for large hypothetical element sizes (40×40 mm) and gradually increased close to unity for small hypothetical element sizes (4×4 mm). For 40×40 and 20×20 mm elements, the fraction of the unaberrated waveform was statistically different that of from 4×4 mm (α=0.05).

However, with the smaller element size, the cost increase can be substantial considering the increase in the channel number of driving electronics, and the fabrication process becomes more challenging. For example, reducing the element size increases the number of elements needed to efficiently pack the surface area of the hemisphere array. Assuming a 75% packing efficiency, it would take over 4000 5×5 mm elements to pack a 300 mm diameter hemisphere. Although a larger element count improves other aspects like the steer-ability of the transducer, it also increases the number of channels needed in the driving electronic system, the amount of cable connecting the transducers to the drive system and the overall footprint of the system. An element size of 17×17 mm was chosen for the final design as it kept the total number of elements reasonable (i.e., a few hundred elements can easily be made in-house) and from a standpoint of phase aberration, should provide comparable performance with elements of 8×8 mm.

### Modules

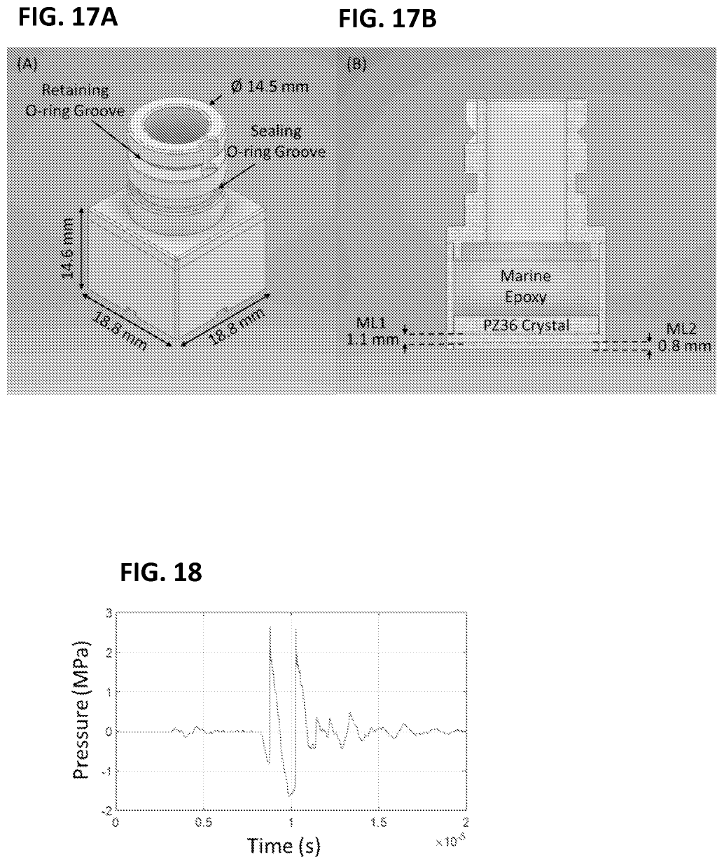

FIGS. 17A-17B illustrate one embodiment of the array modules for the transcranial array application. The housing of the individual transducer modules can be plastic printed with stereolithography, for example. The modules can be designed with an O-ring retaining groove that allows them to be secured to the array scaffold via an easily removable O-ring. This allows modules to be easily replaced with a catheter holder with potential for the arbitrary placement of the catheter hydrophone to be inserted into the brain for evacuation of a liquefied blood clot.

Additionally, in some embodiments, the modules can include a second O-ring groove intended to seal the hemisphere scaffold and allow it to be filled with an acoustic coupling fluid.

### Manufacturing Methods

In one implementation, a 17×17 mm, 700 kHz, PZ36 square can be potted into the plastic module housing using a high-strength epoxy and backed marine-grade epoxy. PZ36 is a soft piezoelectric material. The marine epoxy backing ensures a water-tight seal around the PZ36 element that allows the module to be fully submersed. The transducers can be fabricated with a dual matching layer to improve transmission into the water based propagating media. The acoustic properties of the PZ36 and matching layers used in the transducer design are shown in Table 1.

TABLE 1

| | Acoustic Impedance | Sound Speed |
|---|---|---|
| Material | (MRayI) | (m/s) |
| PZ36 | 17.6 | 2842 |
| PerFORM (ML1) | 5.3 | 3200 |
| PA 12 White (ML2) | 2.2 | 2150 |

Performance of the Transducer Modules: The pressure waveform produced from a single module at 150 mm for a peak drive voltage of 3 kV is shown in FIG. 18. The acoustic waveform is a short (˜1.5 cycle) pulse with a single large negative cycle.

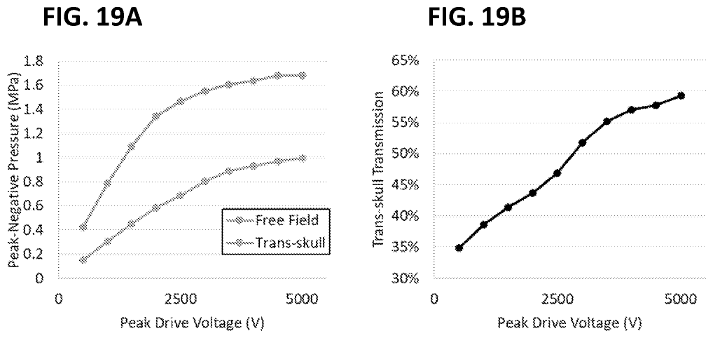

The output of the modules at 150 mm (the focal distance of the array) as a function of drive voltage in the free field and through the human skull is shown in FIG. 19A. At a peak drive voltage of 5 kV, a single module is capable of generating a peak-negative pressure of just over 1.6 MPa in the free field and just about 1 MPa through the skull. The trans-skull transmission of a single module through the skull was plotted as a function of the drive voltage (FIG. 19B). The plot suggests, that as the transducer is driven harder, the trans-skull transmission increases. Driving the transducer at just 500 V results in a trans-skull transmission of 35% while driving the transducer at 5 kV results in a trans-skull transmission of just about 60%. This change in transmission is likely due to a reduction of the non-linear effects of waveform propagation through the skull compared to that of the free-field.

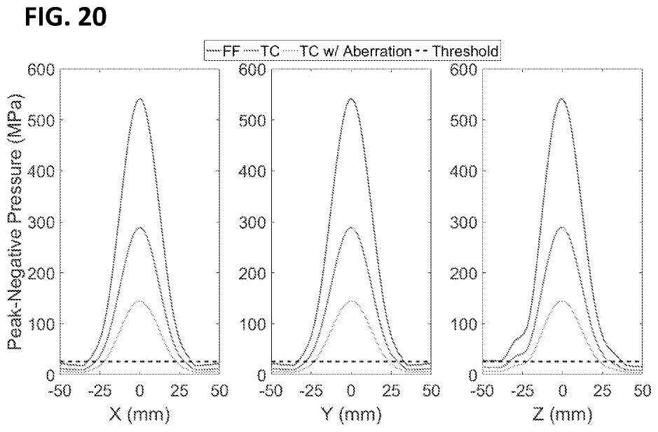

FIG. 20 illustrates the peak-negative pressure output of the 360 element hemisphere array transducer simulated across a ±50 mm electronic focal steering range. The simulation was calibrated to the output of a single 17×17 mm element driven at 3 kV in the free field (FF) and through the skull (TC=transcranial). Aberration through the skull was simulated via a two-fold reduction in the TC steering profile. The dashed line indicates the histotripsy intrinsic threshold (26 MPa).

TABLE 2
The 3-axis steering range and above threshold
volume for different simulation conditions.

| | | Steering Range | | | Volume |
|---|---|---|---|---|---|
| | | X (mm) | Y (mm) | Z (mm) | (mL) |
| FF | −3 dB | 29 | 29 | 29 | — |
| | >26 MPa | 65 | 67 | 78 | 177 |
| TC | −3 dB | 29 | 29 | 29 | — |
| | >26 MPa | 55 | 57 | 64 | 105 |
| TC w/ | −3 dB | 29 | 29 | 29 | — |
| Aberration | >26 MPa | 45 | 46 | 45 | 48 |

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A histotripsy transducer array, comprising:
a scaffold; and
a plurality of transducer elements arranged on the scaffold such that the ultrasound transducer array has a packing density greater than or equal to 90%, wherein the array is configured to transmit histotripsy pulses at an amplitude exceeding a cavitation threshold of one or more bubble cloud locations in a target tissue.

2. The histotripsy transducer array of claim 1, wherein the scaffold is concave.

3. The histotripsy transducer array of claim 1, wherein the scaffold has a generally rectangular shape.

4. The histotripsy transducer array of claim 1, wherein some of the plurality of transducer elements are arranged within a central region of the scaffold in a plurality of concentric rows of transducer elements.

5. The histotripsy transducer array of claim 1, wherein some of the plurality of transducer elements are arranged in one or more peripheral regions of the scaffold adjacent to the central region.

6. The histotripsy transducer array of claim 1, wherein the transducer elements in the one or more peripheral regions are arranged in arced rows of transducer elements.

7. The histotripsy transducer array of claim 1, further comprising an opening in the scaffold to accommodate an imaging probe disposed within the central region.

8. The histotripsy transducer array of claim 1, wherein the plurality of transducer elements each have the same surface area.

9. The histotripsy transducer array of claim 1, wherein the plurality of transducer elements have varying surface areas.

10. The histotripsy transducer array of claim 1, wherein the plurality of transducer elements are separated by a layer of epoxy less than or equal to 125 microns thick.

11. The histotripsy transducer array of claim 1, wherein the scaffold includes a geometry optimized for transmission of ultrasound waves into a target tissue within the patient's abdomen.

12. The histotripsy transducer array of claim 1, wherein the scaffold includes a geometry optimized for transmission of ultrasound waves into a target tissue within the patient's skull.

13. The histotripsy transducer array of claim 12, wherein the scaffold is hemispherical.

14. A method of designing a transducer array geometry for a target tissue, comprising:
acquiring a plurality of images of a target tissue;
generating an image segmentation mask for each of the plurality of images corresponding to the target tissue;
generating a 3D model of the target tissue from the image segmentation masks;
projecting rays from a point in the 3D model corresponding to the target tissue to distributed discrete points on a sphere to identify available acoustic windows;
summing acoustic windows and excluding regions where less than a threshold value is common across the summed acoustic windows.

15. The method of claim 14, wherein acquiring the plurality of images comprises capturing the plurality of images in real-time.

16. The method of claim 14, wherein acquiring the plurality of images comprises acquiring the plurality of images from a database of historical images.

17. The method of claim 14, wherein the threshold value comprises 80%.

18. The method of claim 14, further comprising segmenting the common aperture into a plurality of nesting arc segment-shaped elements.

19. The method of claim 18, wherein each of the nesting arc segment-shaped elements have an approximately equal aspect ratio and surface area.
